(19) United States
(12) Patent Application Publication
(10) Pub. No.: US 2010/0167283 A1
(43) Pub. Date: Jul. 1, 2010

(54) Analyte Detection

(76) Inventors: Adrian Martin Horgan, Kent (GB); Graham John Worsley, Bedfordshire (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/438,159

(22) PCT Filed: Sep. 6, 2007
(86) PCT No.: PCT/GB2007/003357

§ 371 Date: Nov. 19, 2009

(30) Foreign Application Priority Data
Sep. 8, 2006 (GB) 0617730.7

(51) Int. Cl. C12Q 1/68

(52) U.S. Cl. 435/6

(57) ABSTRACT

A method of detecting an analyte in a sample, comprises the steps of: a) contacting the sample with a first ligand which binds specifically to the analyte and which is immobilised either on, or in the vicinity of, a sensor; b) prior to step (a) contacting the sample, or subsequent to step (a) contacting the immobilised analyte, with a material including a second ligand which binds specifically to the analyte, the material being activatable to form a polymerisation initiator; and c) activating the material; wherein the polymerisation initiator interacts with the sensor to change its physical properties, which causes a change in the optical or acoustic properties of the sensor.

### FIELD OF THE INVENTION

The present invention concerns methods of detecting an analyte in a sample and a kit which can be used to carry out such a method.

### BACKGROUND TO THE INVENTION

It is often important to be able to detect analytes that bind to specific binding sites, such as antigens or spores, when the analyte is present at a very low level. This is particularly the case in the fields of medicine, diagnostics, product safety and security. It is, therefore, advantageous to amplify a signal caused by binding of the analyte.

Detection and amplification methods have been proposed in WO2005/024386 and WO2006/031248 which involve allowing a molecular recognition event to take place thereby forming a complex, linking a photoinitiator label to the complex, contacting the photoinitiator-labelled complex with a polymer precursor and irradiating the complex and the polymer precursor thereby forming a polymer which is detectable. In these methods it is preferred that the polymer formed is, itself, detectable by being fluorescent, magnetic, radioactive or electrically conductive or is formed in such large quantities that it can be detected visibly. Alternatively, the polymer can be detected using an extra step wherein it is swollen with a solution that is fluorescent, magnetic, radioactive or electrically conducting. WO2005/024386 and WO2006/031248 are incorporated herein by reference.

These systems work well, but it would be desirable to increase the amplification further and provide a method that gives more flexibility in terms of the polymer precursors that can be used or does not require polymer precursors at all.

### SUMMARY OF THE INVENTION

According to a first aspect of the invention, a method of detecting an analyte in a sample, comprises the steps of:
a) contacting the sample with a first ligand which binds specifically to the analyte and which is immobilised either on, or in the vicinity of, a sensor;
b) prior to step (a) contacting the sample, or subsequent to step (a) contacting the immobilised analyte, with a material including a second ligand which binds specifically to the analyte, the material being activatable to form a polymerisation initiator; and
c) activating the material;
   wherein the polymerisation initiator interacts with the sensor to change its physical properties, which causes a change in the optical or acoustic properties of the sensor.

According to a second aspect of the invention, a kit for use in a method of detecting an analyte according to the first aspect of the invention, comprises a first ligand which is capable of binding specifically to the analyte, and a sensor, wherein the first ligand is immobilised either on the sensor or on a substrate which is positioned during use in the vicinity of the sensor, and a material including a second ligand which is capable of binding specifically to the analyte, the material being activatable to form a polymerisation initiator.

The present invention makes use of a sensor to detect the presence of the analyte via the presence of the activatable material. The polymerisation initiator produced by activating the activatable material interacts directly or indirectly with the sensor to change its physical properties, for example to make it swell or contract. This interaction alters the optical or acoustic properties of the sensor, and this change is detected.

Due to the use in the present invention of a sensor, there is no need for the polymer itself to be independently detectable. Using a sensor means that greater amplification can be obtained than in methods in which the polymer created is directly detected.

Various different kinds of sensor can be used in the invention, the main criterion for suitability is sensitivity to a change in the physical properties of the sensor. One class of suitable sensors is those that rely on diffraction effects, such as sensors that simply have a surface relief, or those that include a hologram or crystal colloidal array. Alternatively, it is possible to use acoustic sensors, for example those that rely on a resonating quartz crystal.

In a simple embodiment, the polymerisation initiator, which often is or includes free radicals, interacts directly with the sensor to cause it to change its physical characteristics, usually to cause rapid swelling. This embodiment involves few steps and means that sensors can be provided with a very simple construction, without the need for polymer precursors.

In a preferred embodiment, the polymerisation initiator interacts indirectly with the sensor. This occurs when the polymerisation initiator interacts with polymer precursors in the sensor to cause polymerisation, which has an effect on the physical structure of the sensor. This can be achieved by attaching polymer precursor to the sensor itself, or by contacting the sensor with a solution containing polymer precursor. When the polymer precursor is added in a separate step, this usually takes place between steps b) and c), i.e. directly before activation.

The method of the present invention is very sensitive and can be used to detect low levels of analyte as each species of analyte which is present results in the binding of activatable material which, following activation, can cause a much greater response in the sensor than would be caused by a single binding event. Each unit of activatable material which is present can lead, following activation, to the polymerisation of around 10⁶ polymer precursors so the amplification effect is massive.

Activatable material which is suitable for use in the present invention is relatively economical to use and even where polymer precursors are used, these are inexpensive. This is particularly the case compared to labels such as enzymes which are themselves expensive and which require expensive substrates. Furthermore, activatable labels are typically less bulky than enzymes.

In a preferred embodiment, the sensor is a holographic sensor. Using a holographic sensor to detect the presence of the activatable material, following initiation, gives greater amplification than in the prior art methods as small changes in the physical properties of the matrix can lead to substantial changes in the optical properties of the sensor which can then be detected. It also provides a very simple readout. The hologram can be used with opaque samples and it the read-out is photostable with respect to fluorophores.

Many known holographic sensor systems rely on having a matrix which is chemically sensitive to an analyte so that the presence of an analyte directly causes a detectable change in the physical properties of the sensor. Such sensors have been very successful but the detection limit is relatively high which is a major drawback in some applications and prohibits the use altogether for other applications.

The present invention does not require the matrix to be chemically sensitive to the particular analyte, only to be physically sensitive to interaction with the polymerisation initiator either directly or indirectly through polymer formation. This means that the sensor is easier and more cost-effective to manufacture. It also means that the sensor of the invention provides greatly improved sensitivity compared to prior art holographic sensors because the presence of activatable material, associated with a single analyte species, can start a chain reaction leading to a massive change in the physical properties of the matrix and a corresponding change in the optical characteristics of the sensor.

### DETAILED DESCRIPTION OF THE INVENTION

In a preferred embodiment, the first ligand is immobilised on the sensor itself (i.e. attached directly to the surface of the sensor). However, it is also possible to locate the immobilised first ligand on a separate substrate such as a membrane filter in the vicinity of the sensor so that, following activation, the polymerisation initiator formed can be washed onto and hence come into contact with and can interact with the sensor. In this embodiment, any substrate on which the ligands can be immobilised can be used but a filter membrane, such as one made from nitrocellulose, is preferred. The term “in the vicinity of” means that the substrate is located in physical or fluid contact with the sensor so that the polymerisation initiator, once produced, can interact with the matrix.

The invention makes use of optical or acoustic sensors. Acoustic sensors are known; see, for example, WO01/02857 (the content of which is incorporated by reference). They generally comprise a quartz crystal which is made to oscillate by application of an electric current. The crystal has electrodes positioned thereon which provide the current. The frequency of the oscillations depend on the current applied and can be controlled by the current. The frequency of the oscillations can be detected and small changes can be determined.

The frequency of the oscillations is affected by the binding of molecules at the surface due to the change in mass of the sensor. In this way, such systems are already used to detect analytes directly. WO02/12873 and EP1171769 give examples of such systems and are incorporated herein by reference.

In the present invention, in addition to a first ligand which may be provided on the quartz crystal or in the vicinity there of, a polymer precursor is attached to the surface of the quartz crystal. Additional polymer precursor is added before activation. Hence, on activation, the immobilised and the additional polymer precursors polymerise to form a polymer on the surface of the crystal. Hence, the mass attached to the surface of the crystal is increased which changes the frequency of oscillations. This change in frequency can be detected.

In a preferred embodiment, the sensor is an optical sensor. Optical sensors are known in the art and various different kinds are suitable for use in the present invention.

The optical sensor generally comprises a matrix and the sensitive element located therein or thereon. The matrix is usually a hydrogel which can be formed by polymerisation of a hydrophilic monomer which may be natural or synthetic. Suitable materials for use as a matrix (also called support medium) and methods of forming them are disclosed in, for example WO03/087899, WO99/63408 and WO95/26449. For example, the matrix may be formed from the copolymerisation of (meth)acrylamide and/or (meth)acrylate-derived comonomers. In particular, the monomer HEMA (hydroxyethyl methacrylate) is readily polymerisable and cross-linkable. PolyHEMA is a versatile support material since it is swellable, hydrophilic and widely biocompatible. Other examples of holographic support media are gelatine, K-carageenan, agar, agarose, polyvinyl alcohol (PVA), sol-gels (as broadly classified), hydrogels (as broadly classified) and acrylates. Further materials are polysaccarides, proteins and proteinaceous materials, oligonucleotides, RNA, DNA, cellulose, cellulose acetate, siloxanes, polyimides and polyacrylamides.

The hydrogel can be tailored to the present invention by being particularly sensitive to radicals. For example, additional sensitivity can be achieved if the polymer backbone has a pendant unsaturation. This can be achieved using a monomer such as 3-isopropenyl-α,α-dimethylbenzyl isocyanate (m-TMI), an example of a compound which has an isocyanate group at one end and a vinyl group at the other. Post-derivatisation of a HEMA hydrogel involves reaction of hydroxyl groups with the isocyanate of m-TMI, to provide pendant vinyl groups, which cross-link in the presence of radicals. In this way, polymer precursors can be attached to the hydrogel matrix which undergo cross-linking, thereby forming a polymer.

It may also be advantageous to use a low level of cross-linking in the hydrogel, which gives a more highly swollen hydrogel that is more sensitive to polymerisation as it contracts more easily. A hydrogel close to its spinodal point (stability between phases) might also be used, to increase sensitivity.

In one embodiment, the optical sensor relies on a viscosity-sensitive fluorescent probe immobilised on the matrix. Such probes are known in the art as described in articles such as Bioorganic Chemistry 33 (2005) 415-425 by M. A. Haidekker et al or Bioorganic Medicinal Chemistry Letters 8 (1998) 1455-1460 by A. Petric et al, the contents of which are incorporated herein by reference.

As the name suggests, viscosity-sensitive fluorescent probes change their optical properties according to the viscosity of the environment. When a polymerisation initiator interacts with matrix directly or indirectly it causes swelling or contraction of the matrix. Hence, the optical characteristics of the attached flourophores are affected in a detectible matter.

Suitable viscosity-sensitive fluorescent probes include 2-(1,1-dicyanopropenyl)-2,6-dimethylaminonaphthalene (DDNP), 4,4-dimethylaminobenzonitrile (DMABN), (7-amino-4-methylcoumarin-3-acetylamino)hexanoic acid (AMCA) and 9-(dicyanovinyl)-julidine-triethylene glycol ester (CCVJ-TEG).

A further sensor that can be used is one which comprises an optical grating or surface relief comprised of a material having a high refractive index, a substrate layer that supports the optical grating, and one or more specific binding substances immobilized on the surface of the optical grating opposite of the substrate layer. A narrow band of optical wavelengths can be reflected from the biosensor or optical device when the biosensor is illuminated with a broad band of optical wavelengths. The binding of the analyte is detected from the wavelength shift of the reflected light. Such sensors are known in the art, for example, as described by B. Cunningham et al in Sensors and Actuators B, 2002, 81, p. 316-328.

Alternatively, the sensor may comprise a matrix having a crystalline colloidal array (CCA) therein. Such sensors are known in the art, for example as described in The Journal of the American Chemical Society, 2003, 125, 3322-3320 by S. A. Asher et al or Analytic Chemistry Vol 75, No 10, May 15, 2003, by V. L. Alexeev et al. Suitable matrixes are as described above with particular examples being set out in the articles referred to which are herein incorporated by reference. A polyacrylamide hydrogel is particularly good matrix for use in this embodiment. The CCA can be made from any colloidal particles such an be stabilised in a hydrogel in this way. Highly charged monodisperse polystyrene colloids have been used for this purpose in the past.

In a similar way to a holographic sensor (discussed below) the embedded CCA diffracts visible light with the wavelength of diffraction being dependant on the volume of the hydrogel. Hence, interaction of free radicals with the hydrogel which results in contraction or swelling will give a detectable change in the optical characteristics of the sensor. Polymerisation within the hydrogel will have a rapid and significant effect on the hydrogel volume which will be signalled by a detectable change in optical characteristics of the sensor.

Another optical sensor that could be used relies on surface plasmon resonance whereby the analyte of interest is detected from a change in the dielectric constant. Such sensors are known in the art, for example, as described by I. D. Parsons et al in Nucleic Acids Res. 1995, 23, 211-216 and Anal. Biochem. 1997, 254(1), 82-87.

In the preferred embodiment, the sensor is a holographic sensor which comprises a matrix that has a hologram therein or thereon. Holographic sensors which may be suitable for use in the present application are disclosed in, for example, WO03/087899, WO99/63408 and WO95/26449, which are incorporated herein by reference.

The matrix is usually a hydrogel which as set out above. Alternatively, gelatin is a standard matrix material for supporting photosensitive species, such as silver halide grains. Gelatin can also be photo cross-linked by chromium III ions, between carboxyl groups on gel strands.

A hologram is recorded in the matrix in any conventional manner. For example, soluble salts, such as silver halide salts, can be perfused into the matrix, and then made to undergo a reaction to form an insoluble sensitive precipitate in which the image is formed, as described in WO99/63408. Alternatively, “silver-free” systems can be formed, as described in WO04/081676.

The hologram in the sensor of the invention can be generated by the diffraction of light. The hologram may only be visible under magnification, or may be viewable under white light, UV light or infra-red radiation or under specific temperature, magnetism or pressure conditions. The holographic image is preferably of an object or gives a 2- or 3-dimensional effect.

The sensor may further comprise means for producing an interference effect when illuminated with laser light, preferably wherein the means comprises a depolarising layer.

The change in optical characteristics caused by interaction of the polymerisation initiator with the sensor can be detected by the naked eye or by using a device. The device is preferably selected from the group consisting of an optical reader, a mobile phone, a computer and a digital camera. It is envisaged that any type of computer can be used, such as a laptop, a desktop, or a hand held device such as a personal digital assistant (PDA) which is a personal organizer device.

The change in optical properties should be an obvious and non-ambiguous change in the colour or image of the hologram, preferably in the visible region of the electromagnetic spectrum. This gives an accurate and reliable readout that can be observed by the naked eye. To help ensure that this is achieved, the sensor preferably has an optical filter thereon. The optical filter should cover some or all of the surface (or surfaces) of the sensor which are observed to monitor analyte interaction.

The filter can be a lowpass filter (which allows radiation below a certain wavelength to pass through it), a highpass filter (which allows radiation above a certain wavelength to pass through it), or a bandpass filter (which allows radiation having a wavelength within a certain band, or certain bands in the case of a multi-bandpass filter, to pass through it). Hence, the use of such filters controls the frequency of the light that reaches the sensor. The hologram in a holographic sensor acts like a bandpass reflector so the reflection wavelength of the hologram must be in the region of the filtered light to be transmitted back from the sensor to the observer or detector.

Filters are selected to provide a cut-off point for light of a high or low wavelength or both so can ensure that any response is in a particular range, for example, the visible range. They can be used to distinguish between different responses (for example to different analytes or analyte concentrations) which occur at different wavelength. They can also be used to prevent an ambiguous response if the sensor is used in non-optimal light conditions (for example, with monochromatic light). Optical filters can be specifically engineered to optimise the observed response to a specific analyte.

A transparent substrate is usually used in combination with an optical filter and is positioned between the sensor and the filter. Specular reflections from the filter and the transparent substrate are not observed.

The kit of the invention is suitable for use in agricultural studies, environmental studies, human or veterinary prognostics, theranostics, diagnostics, therapy. As appropriate, the holographic sensor is located on a test strip, chip, cartridge, swab, tube, pipette or a fluid sampling or analysis device.

The first ligands in the invention bind to the analyte to be detected and can be any groups that are suitable for this purpose, for example when the analyte is an antigen, the first ligand can be an antibody. The ligand may be also be nucleic acid probe such as DNA which is used to bind a nucleic acid analyte. cDNA, copy DNA that is synthesized in the lab from mRNA templates, is suitable for use in the invention. Similarly, the second ligand can be any material that specifically binds to the analyte. The second ligand can itself be the activatable material or can be linked, either directly or indirectly to an activatable material.

Normally the second ligand is linked to the activatable material before binding to the analyte but the activatable material can also be linked to the second ligand afterwards, for example the activatable material can be introduced in the final step so that it replaces or becomes linked to a species connected to the second ligand. This is particularly useful where the activatable material is particularly sensitive and needs to be added as the last component to minimise unwanted and premature activation. An example is where the second ligand is a nucleotide that is initially linked to biotin (a water-soluble B-complex which is also known as vitamin H or B₇ with formula C₁₀H₁₆N₂O₃S). Once it has become bound to the analyte and a sandwich with a first ligand formed, avidin, a material that can be photo-activated, is introduced which is a protein (found in egg white) that binds very strongly to biotin.

As the binding is specific between a binding site and a particular analyte, it is possible not only to detect, but also to identify an analyte present in a sample, by testing it against different ligands which can be on different sensors or can be in different areas of the same sensor.

Any analyte can be detected, but the invention is preferably used to detect an analyte that undergoes a molecular recognition event, such as an a species with an antigen such as viruses or cells, a spore, a nucleic acid including RNA or DNA, an enzyme, a peptide, a protein or a drug.

The activatable material can be any material that produces, on activation, a polymerisation initiator, such as a free radical, which can interact to change the physical properties of the sensor directly or via polymerisation. For example, the invention can use an activatable material that undergoes different types of activation, for example: chemical activation, e.g. using TEMED catalyst with ammonium/potassium persulfate; a redox activation system e.g. using tert-butylperbenzoate, iron sulphate, hydrochloric acid or sodium ascorbate; thermal activation, e.g. using V50; or photo-activation, e.g. using 2,2-dimethoxy-2-phenylacetophenone (DMPA), (4-benzoylbenzyl)trimethylammonium chloride (Quantacure BTC), riboflavin or sodium 2-anthraquinonesulfonic acid (AQS).

If a photo-activatable material is used, increased response times and sensitivities can be achieved by using a brighter source with a wavelength range matching the absorption maximum of the photoinitiator. The application of heat can speed up the polymerisation reaction leading to enhanced sensitivity.

The activatable material, alone or when linked to the second ligand, should be soluble in water or common organic solvents in which is it introduced to the system. Examples of such solvents are water, alcohol, DMSO or DMF or mixtures thereof.

The unbound activatable material is generally removed from the system by washing to ensure that any signal obtained is purely as a result of the presence of analyte. A washing step may also take place following addition of the sample to the sensor, to wash away unreacted analyte.

Depending on the material used, activation is carried out in different ways, for example, by irradiation or heating.

Where the invention involves forming a polymer, a polymer precursor may be coupled to the sensor or added in an extra step as detailed above. The polymer precursor can be any monomer that is compatible with the polymerisation initiator and reacts to form a polymer in the sensor in the desired way, for example, acrylamide or m-TMI. Some monomers can be inhibited by oxygen and some inhibitors require oxygen to work (e.g. riboflavin). The monomers and polymerisation initiator can be selected to allows greater control over the polymerisation reaction and storage conditions.

Depending on the polymerisation initiator system used, the polymerisation could proceed via either free radical polymerisation or a ‘living’ polymerisation reaction such as atom transfer polymerisation (ATP). Chain transfer agents can be included to control molecular weight and polymerisation rate.

The present invention results in massive amplification of the response due to an analyte binding event but, in some circumstances, particularly when the analyte is a nucleic acid, it may be desirable to increase the response even further to obtain maximum sensitivity.

In this case, it is possible to replicate the analyte using a known method which then allows for more second ligands to bond and hence more activatable material to be present. In this embodiment, dual amplification occurs, firstly from amplification of the amount of analyte leading to more activatable material becoming bound, and secondly from amplification of each binding event by using activatable material.

Suitable methods of replication include polymerase chain reaction (PCR) or reverse transcriptase PCR, ligase chain reaction, strand displacement amplification, DNA cleavage-based signal amplification or rolling cycle amplification. These methods may involve modified polymerases able to incorporate nucleotides labelled with activatable material or a moiety for binding the activatable material post amplification such as biotin. These methods are known in the art.

Rolling circle amplification is particularly useful in the present invention as it is the most specific and versatile amplification method. This method also involves ligation which gives increased specificity as ligase is more stringent than polymerase to 3′ mismatches.

An example of using a rolling circle replication step in the present invention is as follows: a first ligand such as a cDNA or expressed sequence tag (EST) is immobilised on a sensor; an analyte such as mRNA genetic material from an influenza virus is introduced; a replication ligand, complementary ss-DNA, is introduced; the first ligand and replication ligand are ligated; circular (rolling circle) DNA is introduced; the replication ligand is extended in the presence of the circular DNA; the rolling circle is removed; a second ligand such as ss-DNA is introduced which is linked to photo-activatable material; a polymer precursor is introduced and the system is illuminated, which results in formation of a polymer in the matrix, leading to contraction of the matrix and hence a detectable in the optical characteristic.

### EXAMPLES

Proof of principle experiments have been carried out with a holographic sensor to show that polymerisation causes a detectable response. The Examples use a conventional glucose-sensitive holographic sensor to demonstrate how this invention works. In the Examples, a polymer precursor is introduced followed by an activatable material which is activated. Examples 1 to 3 utilise chemical activation, and Example 4 uses photo-activation.

### Example 1

0.265 g acrylamide and 0.0179 g of N,N′-methylenebisacrylamide crosslinker were dissolved in 652 μl MQ-water. 4.6 μl TEMED catalyst (the TEMED could be chemically attached to the second antibody) was added. The solution was vortexed then added to a cuvette containing a glucose-sensitive hologram (5 mol % bisacrylamide crosslinker, 8 mol % 3-acrylamidophenylboronic acid). After equilibration, 40 μl of a 0.05 g/ml potassium persulfate solution was added to the contents of the cuvette (without stirring) and the response of the hologram monitored. Polymerisation was accompanied by a rapid and sudden contraction of the hologram, noticeable from the decrease in the diffraction peak wavelength. A solid gel was observed in the cuvette at the end of the polymerisation reaction.

### Example 2

### Control

4.6 μl TEMED catalyst (the TEMED could be chemically attached to the second antibody) was added to 652 μl MQ-water. The solution was vortexed then added to a cuvette containing a glucose-sensitive hologram (5 mol % bisacrylamide crosslinker, 8 mol % 3-acrylamidophenylboronic acid). After equilibration, 40 μl of a 0.05 g/ml potassium persulfate solution was added to the contents of the cuvette (without stirring) and the response of the hologram monitored. A slight swelling of the hologram was observed, noticeable from a small increase in the diffraction peak wavelength. No gel formation was observed for this cuvette.

### Example 3

### Repeat with a Control

0.265 g acrylamide and 0.0179 g N,N′-methylenebisacrylamide crosslinker were dissolved in 652 μl MQ-water. 4.6 μl TEMED catalyst (the TEMED could be chemically attached to the second antibody) was added. The solution was vortexed and then added to a cuvette containing a glucose-sensitive hologram (5 mol % bisacrylamide crosslinker, 8 mol % 3-acrylamidophenylboronic acid). After equilibration, 40 μl MQ-water was added to the contents of the cuvette without stirring. After a further equilibration period, 40 μl a 0.05 g/ml potassium persulfate solution was added to the contents of the cuvette and the response of the hologram monitored. Polymerisation was accompanied by a rapid and sudden contraction of the hologram, noticeable from the decrease in the diffraction peak wavelength. A solid gel was observed in the cuvette at the end of the polymerisation reaction.

### Example 4

0.265 g acrylamide and 0.0179 g N,N′-methylenebisacrylamide crosslinker were dissolved in 652 μl MQ-water. The solution was vortexed, then added to a cuvette containing a glucose-sensitive hologram (5 mol % bisacrylamide crosslinker, 8 mol % 3-acrylamidophenylboronic acid). After equilibration, 20 μl of a 2% DMPA solution in DMSO (the DMPA could be chemically attached to the second antibody) was added to the cuvette without stirring. After a further equilibration period under the halogen light source, the cuvette was illuminated with a UV lamp (at 350 nm). UV illumination produced rapid polymerisation accompanied by a rapid and sudden contraction of the hologram, noticeable from the decrease in the diffraction peak wavelength. A solid gel was observed in the cuvette at the end of the polymerisation reaction.

1. A method of detecting an analyte in a sample, the method comprising the steps of:
a) contacting the sample with a first ligand which binds specifically to the analyte and which is immobilised either on, or in the vicinity of, a sensor;
b) prior to step (a) contacting the sample, or subsequent to step (a) contacting the immobilised analyte, with a material including a second ligand which binds specifically to the analyte, the material being activatable to form a polymerisation initiator; and
c) activating the material;
wherein the polymerisation initiator interacts with the sensor to change its physical properties, which causes a change in the optical or acoustic properties of the sensor.

2. The method according to claim 1, wherein the polymerisation initiator is a free-radical initiator that interacts directly with the sensor to change its physical properties.

3. The method according to either claim 1, wherein the sensor includes a polymer precursor and the polymerisation initiator interacts with the polymer precursor to form a polymer in the sensor.

4. The method according to claim 1, additionally comprising the step of contacting the sensor with a polymer precursor, so that the polymerisation initiator interacts with the polymer precursor to form a polymer in the sensor.

5. The method according to claim 1, wherein the first ligand is an antibody or nucleic acid, preferably cDNA.

6. The method according to claim 1, wherein the analyte is an antigen, spore or nucleic acid.

7. The method according to claim 1, wherein the second ligand is an antibody linked to activatable material.

8. The method according to claim 1, wherein the material can be activated by a chemical reaction, a redox reaction, heat or irradiation.

9. The method according to claim 1, wherein the analyte is a nucleic acid and the method additionally involves replicating the analyte between steps a) and b).

10. The method according to claim 9, wherein the replicating comprises a polymerase chain reaction, reverse transcriptase polymerase chain reaction, ligase chain reaction, strand displacement amplification, DNA cleavage-based signal amplification or rolling cycle amplification.

11. The method according to claim 9, wherein nucleotides labelled with the activatable material are used with modified polymerases.

12. The method according to claim 1, wherein the sensor further comprises means for producing an interference effect when illuminated with laser light.

13. The method according to claim 12, wherein the means comprises a depolarising layer.

14. The method according to claim 1, wherein the sensor has an optical filter thereon.

15. The method according to claim 14, wherein the optical filter is a bandpass filter.

16. The method according to claim 1, wherein the change in optical or acoustic characteristics is detected using a device selected from the group consisting of an optical reader, a mobile phone, a computer and a digital camera.

17. The method according to claim 1, wherein the sensor is a holographic sensor comprising a matrix having a hologram therein or thereon.

18. The method according to claim 1, wherein the sensor comprises a matrix having a crystalline colloidal array therein.

19. The method according to claim 1, wherein the sensor comprises a matrix having immobilised thereon a viscosity-sensitive fluorescent probe.

20. The method according to claim 1, wherein the sensor comprises a matrix having surface relief.

21. The method according to claim 17, wherein the matrix is a hydrogel.

22. The method according to claim 1, wherein the sensor comprises a quartz crystal resonator having electrodes attached thereto and a polymer precursor immobilised thereon, and the method additionally comprises the step of contacting the sensor with additional polymer precursor, so that the polymerisation initiator interacts with immobilised and additional polymer precursors to form a polymer immobilised on the resonator.

23. The method according to claim 17, wherein the hologram is only visible under magnification.

24. The method according to claim 17, wherein the holographic image is of an object or gives a 2- or 3-dimensional effect.

25. The method according to claim 17, wherein the hologram is viewable under white light, UV light or infra-red radiation.

26. The method according to claim 17, wherein the hologram is viewable under specific temperature, magnetism or pressure conditions.

27. The method according to claim 17, wherein the hologram is generated by the diffraction of light.

28. A kit for use in a method of detecting an analyte wherein said kit comprises a first ligand which is capable of binding specifically to the analyte, and a sensor, wherein the first ligand is immobilised either on the sensor or on a substrate which is positioned during use in the vicinity of the sensor; and a material including a second ligand which is capable of binding specifically to the analyte, the material being activatable to form a polymerisation initiator.

29. The kit according to claim 28, wherein the sensor has a polymer precursor coupled thereto.

30. The kit according to claim 28, which additionally comprises a polymer precursor.

31. The method according to claim 1, used in agricultural studies, environmental studies, human or veterinary prognostics, theranostics, diagnostics or therapy.

32. The kit according to claim 28, wherein the sensor is located on a test strip, chip, cartridge, swab, tube, pipette or a fluid sampling or analysis device.

33. The kit according to claim 28, wherein the sensor is a holographic sensor comprising a matrix having a hologram therein or thereon.
